(19) United States
(12) Patent Application Publication
(10) Pub. No.: US 2012/0301546 A1
(43) Pub. Date: Nov. 29, 2012

(54) ACID-RESISTANT SOFT GEL COMPOSITIONS

(76) Inventor: Emadeldin M. Hassan, Huntington Beach, CA (US)

(21) Appl. No.: 13/116,943

(22) Filed: May 26, 2011

(51) Int. Cl. A61K 9/36; A61K 47/42; A61K 47/38; A61K 47/36; A61K 47/32; A61K 9/14; A61K 9/00; A61K 38/02; A61K 38/13; A61K 31/7034; A61K 31/58; A61K 31/573; A61K 31/4439; A23L 1/30; A23L 1/302; A23L 1/303; A23L 1/304; A61K 47/26

(52) U.S. Cl. 424/465; 514/777; 514/774; 514/781; 514/778; 514/772.6; 424/493; 424/400; 514/1.1; 514/20.5; 514/29; 514/40; 514/174; 514/179; 514/338; 426/103; 426/89; 426/72; 426/73; 426/658

(57) ABSTRACT

The present disclosure describes a delivery device for administration of nutraceuticals or pharmaceuticals, which device contains a soft gel shell comprising a gelatin-based water soluble film forming polymer, an acid insoluble polymer, and at least one reducing sugar and water, including processes, gel mixtures used for device production, and coatings containing such gel mixtures.

### BACKGROUND OF THE INVENTION

1. Field of the Invention

This disclosure relates generally to a delivery device for oral administration of nutraceuticals and/or pharmaceuticals, and specifically to a soft gel capsule comprising a gelatin-based water soluble film forming polymer, an acid insoluble polymer, at least one reducing sugar and water, including processes and gel mixtures used for device production.

2. Background Information

The advent of combinatorial chemistry and high throughput screening (HTS) has resulted in the identification of many highly potent new chemical entities (NCEs) that usually have less than desirable physicochemical properties; i.e., high molecular weight, high lipopholicity (log P), and low aqueous solubility. Poor aqueous solubility has been identified as the single largest physicochemical challenge for the oral absorption of compounds and almost inevitably leads to their lower oral bioavailability from the conventional dose forms.

When a compound demonstrates sufficient solubility in a pharmaceutically acceptable non-aqueous vehicle, soft gel capsules may be useful to deliver the solution as a solid dosage form. The availability of a compound formulated in a soft gel for absorption depends on the initial dissolution and rupture of the soft gel shell and subsequent release and dissolution of its fill contents in the gastrointestinal tract (GIT) fluids. Problems in the dissolution and rupture of the soft gel shell become apparent when exposed to chemical substances such as aldehydes and ketones (e.g., when glucose, fructose, or other aldose sugars are included in drug formulations). These problems are attributed to cross-linking of gelatin (pellicle formation) that causes the gelatin shell to become swollen, tough, rubbery, and insoluble in water. Films formed from gelatin treated with such chemical substances are mechanically not flexible (i.e., prevents or restricts gelatin molecular movement), seal poorly, and more readily be brittle. Further, water insoluble gelatin films act as a barrier (e.g., limits intermolecular penetration of small molecules such as water and standard plasticizers), thus restricting drug release.

Given the cross-linking effects of aldehydes and ketones on gelatin, the use of reducing sugars has routinely been discouraged. To avoid pellicle formation, gelatin is usually plasticized with non-reducing polyols such a glycerin or with non-reducing sugars such as xylitol, sorbitol, and maltitol.

Enteric soft gel capsules are ideally suited for compounds that are acid labile, susceptible to degradation in the stomach, or tend to cause gastric irritation, unacceptable mouth odor, or gastric reflux associated with odor-causing liquids, such as fish or garlic oil. They are also useful with compounds targeted for release in the small intestine, such as for gastrointestinal diseases.

Traditionally, enteric soft gels are prepared by coating with enteric polymers using typical coating technology. Coating has its own disadvantages such as unsuccessful adhesion of the enteric polymer onto the soft gelatin shell due to the shell's inherent flexible nature. This can lead to chipping and peeling of the coat. Enteric coating also results in a hazy and opaque appearance of the capsule and requires an additional step of manufacturing.

The idea of introducing an acid resistant soft capsule shell without coating was described in U.S. Pub. No. 20060165778, where an acid insoluble polymer was combined with a water soluble film forming polymer in the presence of an alkaline aqueous solvent. This binary system resulted in acid resistant capsule shells. Alternate approaches also include tertiary systems as well (see, e.g., U.S. Pub. No. 2007008046 and U.S. Pat. No. 7,041,315).

Whether manufactured using binary or tertiary systems, the efficacy of enteric soft gelatin capsules is primarily dependent on the elasticity of gelatin to blow-fill the soft body and on the gelatin low melting point, including the cohesive nature of the film to cause capsule sealing. In general, the non-gelatin component(s) of the shell composition produces an acid resistant film matrix; however, these components reduce overall elasticity and can therefore deteriorate the sealing of the soft capsules.

Regardless of the method used, the fact that the gelatin is in a water/acid soluble form requires the capsule shell to contain high concentrations of the acid-insoluble, water insoluble, non-gelling polymers, such as cellulosic derivatives or acrylic acid co-polymers, to avoid matrix deterioration as a result of dissolution of the water soluble gelatin. High concentrations of the enteric polymer negatively affect film elasticity and are more expensive than gelatin. Further, the use of hydrophilic or water soluble polysaccharides such as pectin or alginates as the acid resistant polymer is basically limited because of the high viscosity of these polymer solutions, which prevent having high solid content in the gel mass and/or cause poor gel mass flow under gravity, slowing down manufacturing due limited encapsulation speed, and may require additional equipment to deliver the gel mass under pressure to the encapsulation machine. Moreover, highly gelling polymers such as pectin or alginate have a tendency to expand in acid media due to the relatively low solid content of the gel mass. The expansion of the gelling enteric polymer when capsules are exposed to an acidic environment can result in capsule expansion, which weakens the capsule shell structure, especially at the capsule seams, or may delay gastric emptying time because of the enlarged capsule size. Such expansion can lead to prolonged exposure to the gastric acidic environment, to include structural failure of the capsule; this is especially acute when stomach content and movement act as additional mechanical stressors.

What is needed is a cost effective, clear, non-coated soft gel matrix dosage form where potential dosage form failure is greatly diminished, and where the form does not sacrifice gel mass elasticity when wet and is more stable and mechanically stronger after drying.

### SUMMARY OF THE INVENTION

The present disclosure describes a soft gel capsule comprising a gelatin-based water soluble film forming polymer, an acid insoluble polymer, and at least one reducing sugar, including processes and gel mixtures used for pharmaceutical- and nutraceutical-delivery device production.

In embodiments, a soft gel shell matrix is disclosed including a water soluble film former; an acid insoluble polymer; a reducing sugar; and optionally, a plasticizer, where the soft gel shell matrix when dried expands to at least 100% of the original length of the matrix before breaking.

In one aspect, the water soluble film former includes, but is not limited to, gelatin, cellulose derivatives, modified starches, natural or synthetic polymers, and combinations thereof. In a related aspect, the water soluble film former is gelatin. In a further related aspect, the matrix includes about 15 to 55% (w/w), about 18 to 45% (w/w), or about 20 to 40% (w/w) gelatin. In another related aspect, the matrix includes about 25% (w/w) gelatin.

In one aspect, the reducing sugar is glucose, fructose or a combination thereof. In a related aspect, the matrix includes a reducing sugar at about 0.5 to 20% (w/w), at about 2 to 15% (w/w), or at about 3 to 10% (w/w). In a further related aspect, the reducing sugar is fructose. In another related aspect, the matrix includes about 3.75% (w/w) fructose.

In another aspect, the acid insoluble polymer includes cellulose acetate phthalate (CAP), cellulose acetate trimellitate (CAT), cellulose acetate succinate (CAS), hydroxypropyl methyl cellulose phthalate (HPMCP), hydroxypropyl methyl cellulose acetate succinate (HPMCAS), carboxy methyl ethyl cellulose (CMEC); copolymers of methacrylic acid and methyl methacrylate, ethyl acrylate; terpolymers of methacrylic acid, methacrylate, ethyl acrylate, polyvinyl acetate phthalate (PVAP), pectin and alginic acid derivatives, and combinations thereof.

In one aspect, the plasticizer includes sorbitol, glycerol, polyethlene glycol, poly-alcohols with 3 to 6 carbon atoms, citric acid, citric acid esters, triethyl citrate, and combinations thereof.

In another aspect, the matrix includes a water soluble film former at about 15 to 55% (w/w), an acid soluble polymer at about 0.5 to 30% (w/w), a reducing sugar at about 0.5 to 20% (w/w), a plasticizer at about 5 to 30% (w/w), and about 10 to 40% (w/w) water.

In one aspect, the matrix includes additives including flavors, sweeteners, colorants, preservatives, fill, and combinations thereof, where the matrix possesses elastic properties such that the matrix forms a soft gel capsule via rotary die technology.

In embodiments, an acid resistant soft gel capsule is disclosed including a water soluble film former; an acid insoluble polymer; a reducing sugar; a plasticizer; water and a fill, where the fill includes a pharmaceutical composition or a nutraceutical composition.

In one aspect, the gelatin has between about 50 to 275 blooms. In another aspect, the reducing sugar is fructose or glucose. In a related aspect, the matrix includes fructose at between about 0.5 to 20% (w/w). In a further related aspect, the matrix includes about 3.75% (w/w) fructose.

In another aspect, the pharmaceutical composition includes diclofenac sodium, hydroxyethyl pyrrolidine diclofenac, diethylammine diclofenac, ibuprofen, flurbiprofen, ketoprofen, idomethacin, mefenamic acid, naproxene, nimesulide, piroxicam, amiodarone, diisopyramide, verapamil, propranolol, amoxicillin, flucloxacillin, gentamicin, rifampicin, erythromicine, cephalosporin, amfotericin, buconazol nitrate, ketoconazol, econazol, fluconazole, flucitosina, griseofulvine, itraconazol, miconazol, rystatin, sulconazol, tioconazol, acyclovir, gancyclovir, AZT, protease inhibitor, amlodipine, clonidine, diltiazem, felodipine, guanabenz acetate, isradipine, minoxidil, chloride nicardipine, nifedipine, chloride prazosin, papaverine, carbamazepine, difenidramine, chlorfeniramine, chlorciclizine, prometazin, acrivastine, loratadine, terfenadine, cyclosporine, decarbazine, etoposide, lomustine, melphalan, mitomicin, mitoanthrone, procarbazine, taxol and derivatives thereof, alprazolam, bromazepam, diazepam, lorazepam, oxazepam, temazepam, sulpiride, triazolam, alprenolol, atenolol, oxprenolol, pindolol, propranolol, salbutamol, salmeterol, amrinone, digitoxinn, digoxin, lanatoside C, medigoxine, ubidecarenone, bechlomethasone, betamethasone, budesonide, cortisone acetate, desossimetasone, dexamethasone, fludrocortidone acetate, flunisolide, hydrocortisone, methylprednisolone, prednisone, triamicinolone, cimetidine, cisapride, domperidone, famotidine, loperamide, mesalazine, omeprazol, ondansetron, ranitidine chloride, bezafibrate, chlorofibrate, gemfibrozil, probucol, amil nitrate, gliceryltrinitrate, isosorbide dinitrate and mononitrate, pentaeritritol tetranitrate, Nicotina, codeine, destropropoxifene, dihydrocodeine, morphine, pentazocine, methadone, danazol, ethynilestradiol, medroxyiprogesterone acetato, methyltestosterone, testosterone, noretistrone, norgestrel, estradiolo, estriolo, progesterone, stilbestrolo, diethylstilbestrol, peptides, peptidic molecules, and combinations thereof.

In another aspect, the nutraceutical composition includes material or material extracts from artichoke, bilberry, bioflavonoid, boswella, bupleurium, chamomile, chlorophyll, cranberry, damiana, echinacea, essiac, garcinia cambogia, garlic, germanium, ginger, gingko, ginseng, goldenseal, grape seed, green tea, hawthorne berry, hesperidin, hops, horse chestnut hydrangea, hypericum, indole-3-carbinol, licorice, lycopene, nettle root, peppermint, periwinkle, policosanol, psyllium, pygeum, quercetin, raspberry, resveratol, rutin, sassafras, saw palmetto, silymarin, tribulus terestris, turmeric, valerian, wild yarn, acetyl-1-carnosine, alpha lipoic acid, amylase, androstendiol, androstendione, arginine, ascorbic acid, B vitamin, β-carotene, biotin, bromelain, calcium, chicken collagen, chitosan, choline, chondroitin, coenzyme Q10, creatine, dehydroepiandrosterone, diethylmethylaminoethanol, dihydroepiandsterone, dimethylglycine, DMSO, gammahydroxybutric acid (GABA), glucosamine, glutamine, glutathione, hyaluronic acid, hydroxytryptophan, indium, isoleucine, 1-carnitine, lactoferrin, lecithin, leucine, lipase, lumbrokinase, lutein, magnesium, melatonin, Methylcobalamin, methylsulfonylmethane, MGN 3, ornithine, pancreatin, panthethoic acid, papain, para-amino benzoic acid (PABA), phenylalanine, phosphatidylcholine, phosphatidylserine, potassium, pregnenalone, protease, retinoic acid, retinol, s-adenosyl-methionine, selenium, taurine, theanine, thymase, tocopherol, trimethylglycine, tryptophan, tyrosine, valine, vinpocetine, vitamin D, vitamin A, zeathanthine, zinc and combinations thereof.

In embodiments, a process of manufacturing a shell composition into soft capsules is disclosed including preparing a solution comprising a water soluble film former, an acid-insoluble polymer, a reducing sugar and mixing with appropriate plasticizers to form a gel mass; casting the gel mass into films or ribbons using heat-controlled drums or surfaces; and forming a soft capsule using a rotary die device, where the manufactured soft capsule has a burst strength of greater than about 7 to about 55 kg and resists acid stress at about 0.1N HCl for at least one hour. In one aspect, the burst strength is between about 12 to about 18 kg.

In one aspect, the thickness of the films or ribbons is from about 0.015 inches to about 0.050 inches. In another aspect, the thickness of the films or ribbons is about 0.030 inches.

In a further aspect, the manufactured soft capsule disintegrates at pH 6.8 in less than about 6 minutes, less than about 45 minutes, or less than about 20 minutes.

In embodiments, an enteric or delayed release delivery device for administration of a nutraceutical composition, a pharmaceutical composition, food, or food supplement to a subject in need thereof is disclosed, where the device includes glucose or fructose.

In embodiments, a coating is disclosed including a water soluble film former; an acid insoluble polymer; a reducing sugar; and optionally a plasticizer; where the reducing sugar does not render the resulting film un-sealable. In one aspect, the coating covers a tablet, powder, solid or semi-solid comprising a material that is sensitive to acidic pH and said material is to be released into the intestine. In a related aspect, the reducing sugar is fructose.

### BRIEF DESCRIPTION OF THE DRAWINGS

FIG. 1 shows a plot of gelatin film elasticity as a function of increasing fructose concentration (closed diamonds, with 8% CAP; open squares, without CAP).

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a deliver device” includes one or more delivery devices, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein may be used in the practice or testing of the invention, as it will be understood that modifications and variations are encompassed within the spirit and scope of the instant disclosure.

As disclosed herein, it has been discovered that, despite well known assertions to the contrary, reducing sugars provide unexpected advantages to gelatin films containing acid insoluble polymers both in terms of elasticity and mechanical strength. In embodiments, a reducing sugar is present at about 0.5% to about 25% (w/w), about 2% to about 20% (w/w), or about 3% to about 15% (w/w). In some embodiments, the reducing sugar is present at about 3.75% (w/w) or about 5.5% (w/w). In embodiments, the reducing sugar is glucose or fructose.

While not being bound by theory, the presence of reducing sugars appear to have exceptional plasticizing effect on the gelatin-acid insoluble polymer mixture, but did not render the gel mass un-sealable or rubbery as expected from normal cross-linking reactions associated with known cross-linking agents such as formaldehyde.

As used herein, the term “soft gel” means a one-piece, hermetically sealed soft gelatin shell containing a solution, a suspension, or a semisolid, referred to as fill formulation, fill material, or fill. A fill formulation for encapsulation into soft gels may be a solution, liquid-in-liquid dispersion, or a solid-in-liquid suspension. In embodiments, fill formulation are prepared using standard procedures employed in pharmaceutical solution, suspension, and semisolid manufacturing. In embodiments, the manufacturing process for soft gel production may be the rotary die process (see, e.g., Canadian Pat Nos.: CA333007, CA416849, CA416850, CA422183, CA453243, and U.S. Pat. No. 6,769,226).

As used herein, the term “dosage form” applies to any solid object, semi-solid, or liquid composition designed to contain a specific pre-determined amount (dose) of a certain ingredient, for example an active ingredient as defined below. Suitable dosage forms may be pharmaceutical drug delivery systems, including those for oral administration, buccal administration, rectal administration, topical or mucosal delivery, or subcutaneous implants, or other implanted drug delivery systems; or compositions for delivering minerals, vitamins and other nutraceuticals, oral care agents, flavors, and the like. In embodiments, the dosage may contain liquid or semi-solid components. In embodiments, the dosage form is an orally administered system for delivering a pharmaceutical active/nutritional supplement or nutraceutical ingredient to the gastro-intestinal tract of a human. In other embodiments, the dosage form may be an orally administered “placebo” system containing pharmaceutically inactive ingredients, and the dosage form is designed to have the same appearance as a particular pharmaceutically active dosage form, such as may be used for control purposes in clinical studies to test, for example, the safety and efficacy of a particular pharmaceutically active ingredient.

In embodiments, a soft gel shell matrix is disclosed including a water soluble film former; an acid insoluble polymer; a reducing sugar; optionally a plasticizer, and water. In embodiments, the water may be present at about 10% to about 40% (w/w), about 12% to about 35% (w/w), or about 15% to about 30% (w/w).

Films as disclosed herein may be made by casting a final gel mass that does not dissolve or disintegrate in acids, such as 0.1M hydrochloric acid, despite the fact that the majority of shell ingredients (more than 50%) may dissolve in, or may be miscible with, acids. Enteric films made using the disclosed compositions remain substantially intact in hydrochloric acid. Further, enteric films of the present disclosure may reduce migration of small molecules through them in acidic environments. In embodiments, the final gel mass provides films of increased strength without substantially compromising film elasticity. Moreover, casting films according to the disclosure are able to be sealed at normal temperature range typically used for making soft gel capsules (from about 80° F. to about 105° F.) or may be used to surround or enrobe tablets to make them enteric. The gel masses may also be cast around pins to form two-piece hard capsules as illustrated in Remington, the Science and Practice of Pharmacy, 2005, 21^{st} edition, Lippincott Williams & Wilkins Company, Philadelphia, Pa.

In embodiments, the dissolution characteristics of the delivery device meet USP specifications for enteric dosage form containing an active ingredient. Enteric compositions as envisaged may comprise a film-forming polymer (e.g., gelatin or a synthetic polymer), at least one reducing sugar, and an acid-insoluble polymer, may be used to contain a fill that is liquid, semi-solid, or solid.

Examples of film-former polymers that are useful in this invention may be of natural origin, including but not limited to, gelatin, or of synthetic nature such as hydroxypropyl methyl cellulose. Gelatin is a mixture of water-soluble proteins derived from collagen by hydrolysis. The protein fractions consist almost entirely of amino acids. These amino acids are joined by amide linkage to form a linear polymer varying from 15,000 to 250,000 M_{w}.

Gelatin may be characterized by its mode of manufacture. Type A gelatin (pH 3.8-6.0; isoelectric point 6-8) is derived by acid hydrolysis of animal parts such as pork skin and contributes plasticity and elasticity to the blend. Type B gelatin (pH 5.0-7.4; isoelectric point 4.7-5.3) is derived from basic hydrolysis of animal parts such as bones and animal skin and contributes high gel strength to the blend. Gelatin used in pharmaceutical applications may be a blend of the two types, although sometimes only type A or type B may be used. Various grades of gelatin with differing particle sizes and molecular weight are available commercially in the form of translucent sheets, granules, or powders. Gelatin usually is graded according to gel strength, expressed as bloom strength, which is the weight in grams that, when applied with a 12.7 mm-diameter plunger, will produce a depression exactly 4 mm deep in a matured jelly containing 6.66% w/w gelatin in water. In embodiments, gelatin is limed bone gelatin 150 bloom, and may be present at about 15% to about 55% (w/w), about 18% to about 45% (w/w), or about 20% to about 40%. In some embodiments, gelatin is present at about 28% (w/w).

Acid insoluble polymers are mainly polysaccharide in nature such as modified cellulosic derivatives, pectins and algenic acid, and derivatives. For example, ammonium, potassium, or sodium alkali salts of these polymers can dissolve in water, and will gel or precipitate in acid medium or in the presence of heavy metals such as magnesium and calcium. In embodiments, heavy metal salts of acid insoluble polymers are made during the preparation of the gel shell matrix by adding water soluble salts of calcium or magnesium, such as magnesium or calcium chloride to the formula. Examples of polysaccharide acid-insoluble polymers are cellulose acetate phthalate (CAP), cellulose acetate butyrate, hydroxypropyl methyl cellulose phthalate, pectin, algenic acid salts such as sodium or potassium alginate. Other acid insoluble polymers include shellac, acrylic acid-methacrylic acid copolymers (available under the trade name of EUDRAGIT (Rohm America Inc., Piscataway, N.J.) as powder or 30% aqueous dispersion, or under the trade name of EASTACRYL as 30% dispersion (Eastman Chemical Company, Kingsport, Tenn.), and sodium alginate. Acrylic-methacrylic acid copolymers are stable and may be used in some embodiments. Acid-insoluble polymers specifications are detailed in the United States Pharmacopoeia. In embodiments, an acid insoluble polymer is present at about 0.5% to about 30% (w/w), about 2 to about 25% (w/w), or about 4% to about 20% (w/w). In some embodiments, the amount of acid insoluble polymer is present at about 8% (w/w).

While the presence of acid insoluble polymers provide enteric properties, they have to be present at a relatively high ratio relative to gelatin. For example, in embodiments, concentrations were approximately about 25% to about 30% of gelatin when water soluble polymers such as acrylic acid or carboxylic acid derivatives of cellulose are used. The composition ratio between the film-former and the acid-insoluble polymer may be adjusted so that the gel mass may be made into soft capsules.

Useful plasticizers according to the present disclosure include, but are not limited to, glycerol, sorbitol, polyethylene glycol, citric acid, citric acid esters, such as tri-ethyl citrate, or combinations thereof. In embodiments, the plasticizer is present at about 5% to about 30% (w/w), about 10% to about 28% (w/w), or about 15% to about 25%.

In embodiments, enteric gel masses may be made by dissolving the acid-insoluble polymer powder in aqueous solution containing a basic compound or an alkali. Alkalai such as ammonia, sodium hydroxide, or potassium hydroxide, or liquid amines such as tri-ethanol amine or ethylene di-amine. The amount of alkali may be adjusted to give a final pH value of the gel mass less than or equal to about pH 9.0. In embodiments, the final pH does not exceed 8.5. Volatile alkalis such as ammonia and ethylene di-amine may be used. Basic alkalinizing compounds can be any water soluble chemical whose solution in water has a pH value higher than 7. Examples of basic alkalinizing compounds are tri- and di-sodium or potassium salts of phosphoric acid, of di- or tri-carboxylic acids such as citric or tartaric acid or basic amino acids such as arginine and lysine. The film-former may then be wetted by the plasticizer and mixed with the acid-insoluble gel to make a final homogeneous mix in a heat-controlled vessel and may be degassed by using vacuum. The alkali concentrations may not require an additional step such as heating or neutralizing with acid in order to neutralize the gel mass or the finished capsules.

In embodiments, the enteric gel may be made by using a ready-made aqueous dispersion of the acid-insoluble polymer by adding alkaline materials such as ammonium, sodium, or potassium hydroxides or other alkalis that will cause the acid-insoluble polymer to dissolve, such as triethanol amine or ethylene di-amine or a combination thereof. The plasticizer-wetted, film-forming polymer may then be mixed with the solution of the acid-insoluble polymer. In embodiments, acid-insoluble polymers in the form of salts of the above-mentioned bases or alkalis may be dissolved directly in water and mixed with the plasticizer-wetted, film-forming polymer.

Further, additives such as excipients for providing preparations (for example, lactose, sucrose, D-mannitol, erythritol, multitol, trehalose, sorbitol, corn starch, potato starch, wheat starch, rice starch, crystalline cellulose, silicic acid anhydride, calcium metaphosphorate, sedimented calcium carbonate, calcium silicate, and the like), polymers such as hydroxypropyl cellulose, hydroxypropyl methylcellulose, polyvinyl pyrrolidone, methyl cellulose, polyvinyl alcohol, carboxymethyl cellulose sodium, partial a starch, a starch, sodium alginate, pullulan, gum arabic powder and the like, disintegrants (for example, low substituted hydroxypropyl cellulose, carmelose, carmelose calcium, carboxymethylstarch sodium, cross carmelose sodium, crosspovidon, hydroxypropylstarch and the like), flavoring agents (for example, citric acid, ascorbic acid, tartaric acid, malic acid, aspartame, acesulfam potassium, thaumatin, saccharin sodium, glycylrrhizin dipotassium, sodium glutamate, sodium 5′-inosinate, sodium 5′-guanylate and the like), surfactants (for example, poloxemers, polysolvate (polysolvate 80 and the like), polyoxyethylene-polyoxypropylene copolymer, sodium laurylsulfate and the like), perfumes (for example, lemon oil, orange oil, menthol, peppermint oil and the like), lubricants (for example, magnesium stearate, sucrose fatty acid eater, sodium stearylfumarate, stearic acid, talc, polyethylene glycol and the like), colorants (for example, titanium oxide, edible Yellow No. 5, edible Blue No. 2, iron (III) oxide, yellow iron (III) oxide, and the like), antioxidants (for example, sodium ascorbate, L-cysteine, sodium bisulfate, and the like), masking agents (for example, titanium oxide and the like), and antistatic agents (for example, talc, titanium oxide and the like) may be used.

Neutraceuticals may include, but are not limited to, minerals, vitamins or food supplements from plant, animal, insect, micro-organism or synthetic origin such as materials, oils, or extracts from artichoke, bilberry, bioflavonoid, boswella, bupleurium, chamomile, chlorophyll, cranberry, damiana, echinacea, essiac, garcinia cambogia, garlic, germanium, ginger, gingko, ginseng, goldenseal, grape seed, green tea, hawthorne berry, hesperidin, hops, horse chestnut hydrangea, hypericum, indole-3-carbinol, licorice, lycopene, nettle root, peppermint, periwinkle, policosanol, psyllium, pygeum, quercetin, raspberry, resveratol, rutin, sassafras, saw palmetto, silymarin, tribulus terestris, turmeric, valerian, wild yarn, and may also include acetyl-1-carnosine, alpha lipoic acid, amylase, androstendiol, androstendione, arginine, ascorbic acid, B vitamin, β-carotene, biotin, bromelain, calcium, chicken collagen, chitosan, choline, chondroitin, coenzyme Q10, creatine, dehydroepiandrosterone, diethylmethylaminoethanol, dihydroepiandsterone, dimethylglycine, DMSO, gammahydroxybutric acid (GABA), glucosamine, glutamine, glutathione, hyaluronic acid, hydroxytryptophan, indium, isoleucine, 1-carnitine, lactoferrin, lecithin, leucine, lipase, lumbrokinase, lutein, magnesium, melatonin, Methylcobalamin, methylsulfonylmethane, MGN 3, ornithine, pancreatin, panthethoic acid, papain, para-amino benzoic acid (PABA), phenylalanine, phosphatidylcholine, phosphatidylserine, potassium, pregnenalone, protease, retinoic acid, retinol, s-adenosyl-methionine, selenium, taurine, theanine, thymase, tocopherol, trimethylglycine, tryptophan, tyrosine, valine, vinpocetine, vitamin D, vitamin A, zeathanthine, zinc and combinations thereof. In one aspect, nutraceuticals also include volatile or essential oils such as ginger oil, peppermint oil, and parsley oil, or combinations thereof.

Nutraceuticals may also include, but are not limited to essential fatty acids and their natural and synthetic derivatives and esters, oils containing omega fatty acids and their derivatives, including 9,12,15-octadecatrienoic acid (alpha-linolenic acid) [18:3, ω3]; 6,9,12,15-octadecatetraenoic acid (stearidonic acid) [18:4, ω3]; 11,14,17-eicosatrienoic acid (dihomo-alpha-linolenic acid) [20:3, ω3]; 8,11,14,17-eicosatetraenoic acid [20:4, ω3]; 5,8,11,14,17-eicosapentaenoic acid [20:5, ω3]; 7,10,13,16,19-docosapentaenoic acid [22:5, ω3]; 4,7,10,13,16,19-docosahexaenoic acid [22:6, ω3]; 9,12-octadecadienoic acid (linoleic acid) [18:2, ω6]; 6,9,12-octadecatrienoic acid (gamma-linolenic acid) [18:3, 6]; 8,11,14-eicosatrienoic acid (dihomo-gamma-linolenic acid) [20:3, ω6]; 5,8,11,14-eicosatetraenoic acid (arachidonic acid) [20:4, ω6]; 7,10,13,16-docosatetraenoic acid [22:4, ω6]; 4,7,10,13,16-docosapentaenoic acid [22:5]; 6,9-octadecadienoic acid [18:2, ω9]; 8,11-eicosadienoic acid [20:2, ω9]; and 5,8,11-eicosatrienoic acid (Mead acid) [20:3, ω9]. In embodiments, the oil phase comprises at least one fatty acid or fatty acid ester selected from DHA, DTA, EPA, and LA, and esters thereof, the acylglycerols, and triacylglycerols.

Further such oil may be obtained from single cell organism derived oils (e.g., Dhasco or Arasco), fish oils (e.g., cod liver oil, salmon oil, tuna oil, menhaden oil, sardine oil, capelin oil, herring oil, halibut liver oil, shark liver oil, and the like), plant oils (e.g., evening primrose oil, borage oil, blackcurrant seed oil, and the like), plant, animal (including fish) or microorganism oil extracts (e.g., gamma linolenic acid, EPA, DHA or concentrates thereof), synthetic or semi-synthetic oils (e.g., conjugated linoleic acid), oils from genetically modified plants, animals or microorganisms, lipophilic vitamins, and the like. In embodiments, fish oils or fish oil concentrates or extracts are described.

The pharmaceutical compositions as disclosed may have an extended activity of a drug by a release-controlled system where therapeutic effect is revealed for at least about 6 hours, about 8 hours, about 12 hours or about 16 hours.

The active ingredients are not particularly limited, and may be applied irrespective of the region of drug efficacy. Exemplified are anti-inflammatory drugs such as indomethacin and acetaminophen, analgesics such as morphine, cardiovascular agonists such as diazepam and diltiazepam, antihistamines such as chlorophenylamine maleate, antitumors such as fluorouracil and aclarubicin, narcotics such as midazolam, anti-hemostasis agents such as ephedrine, diuretics such as hydrochlorothiazide and furosemide, bronchodilators such as theophyline, antitussives such as codeine, antiarrythmic agents such as quinidine and dizoxin, antidiabetics such as tolbutamide, pioglitazone and troglitazone, vitamins such as ascorbic acid, anticonvulsants such as phenyloin, local anesthetics such as lidocaine, adrenocortical hormones such as hydrocortisone, drugs effective for central nerve such as eisai, hypolipidemic drugs such as pravastatin, antibiotics such as amoxicillin and cephalexin, digestive tract exitomotory agents such as mosapride and cisapride, H₂ receptor blockers such as famotidine, ranitidine and cimetidine which are the remedies of gastritis, symptomatic gastroesophageal reflux disease, and gastric and duodenal ulcers, and benzimidazole proton pump inhibitors (PPI) represented by lansoprazole and optically active isomers thereof (R-isomer and S-isomer), omeprazole, omeprazole salts and optically active isomers thereof (S-isomer: S omeprazole), rabeprazole and optically active isomers thereof, pantoprazole and optically active isomers thereof and the like, and imidazopyridine PPI represented by tenatoprazole and the like.

A wide variety of therapeutically active agents may be used in conjunction with the present delivery device. The therapeutically active agents (e.g., pharmaceutical agents) which may be used in the compositions of the present invention include both water soluble and water insoluble drugs. Examples of such therapeutically active agents include antihistamines (e.g., dimenhydrinate, diphenhydramine, chlorpheniramine and dexchlorpheniramine maleate), analgesics (e.g., aspirin, codeine, morphine, dihydromorphone, oxycodone, and the like), non-steroidal anti-inflammatory agents (e.g., naproxyn, diclofenac, indomethacin, ibuprofen, sulindac), anti-emetics (e.g., metoclopramide), anti-epileptics (e.g., phenyloin, meprobamate and nitrezepam), vasodilators (e.g., nifedipine, papaverine, diltiazem and nicardipine), anti-tussive agents and expectorants (e.g., codeine phosphate), anti-asthmatics (e.g. theophylline), antacids, anti-spasmodics (e.g., atropine, scopolamine), antidiabetics (e.g., insulin), diuretics (e.g., ethacrynic acid, bendrofluazide), anti-hypotensives (e.g., propranolol, clonidine), antihypertensives (e.g., clonidine, methyldopa), bronchodilators (e.g., albuterol), steroids (e.g., hydrocortisone, triamcinolone, prednisone), antibiotics (e.g., tetracycline), antihemorrhoidals, hypnotics, psycho-tropics, antidiarrheals, mucolytics, sedatives, decongestants, laxatives, vitamins, stimulants (including appetite suppressants such as phenylpropanolamine), as well as salts, hydrates, and solvates of the same. In embodiments, pharmaceuticals as envisage include, but are not limited to, diclofenac sodium, hydroxyethyl pyrrolidine diclofenac, diethylammine diclofenac, ibuprofen, flurbiprofen, ketoprofen, idometacina, mefenamic acid, naproxene, nimesulide, piroxicam, amiodarone, diisopyramide, verapamil, propranolol, amoxicillin, flucloxacillin, gentamicin, rifampicin, erythromicine, cephalosporin, amfotericin, buconazol nitrate, ketoconazol, econazol, fluconazolo, flucitosina, griseofulvine, itraconazol, miconazol, rystatin, sulconazol, tioconazol, acyclovir, gancyclovir, AZT, protease inhibitor, amlodipine, clonidine, diltiazem, felodipine, guanabenz acetate, isradipine, minoxidil, chloride nicardipine, nifedipine, chloride prazosin, papaverine, carbamazepine, difenidramine, chlorfeniramine, chlorciclizine, prometazin, acrivastine, loratadine, terfenadine, cyclosporine, decarbazine, etoposide, lomustine, melphalan, mitomicin, mitoanthrone, procarbazine, taxol and derivatives thereof, alprazolam, bromazepam, diazepam, lorazepam, oxazepam, temazepam, sulpiride, triazolam, alprenolol, atenolol, oxprenolol, pindolol, propranolol, salbutamol, salmeterol, aminone, digitoxinn, digoxin, lanatoside C, medigoxine, ubidecarenone, bechlomethasone, betamethasone, budesonide, cortisone acetate, desossimetasone, dexamethasone, fludrocortidone acetate, flunisolide, hydrocortisone, methylprednisolone, prednisone, triamicinolone, cimetidine, cisapride, domperidone, famotidine, loperamide, mesalazine, omeprazol, ondansetron, ranitidine chloride, bezafibrate, chlorofibrate, gemfibrozil, probucol, amil nitrate, gliceryltrinitrate, isosorbide dinitrate and mononitrate, pentaeritritol tetranitrate, Nicotina, codeine, destropropoxifene, dihydrocodeine, morphine, pentazocine, methadone, danazol, ethynilestradiol, medroxyiprogesterone acetato, methyltestosterone, testosterone, noretistrone, norgestrel, estradiolo, estriolo, progesterone, stilbestrolo, diethylstilbestrol, peptides, peptidic molecules, and combinations thereof. The above lists are not meant to be exhaustive.

In embodiments, in addition to providing a matrix for a soft gel delivery device comprising fill components, the matrix-containing films as disclosed may be used as a coating for tablets, granules, a layer for delayed release or to sequester components for admixture function (e.g., multi-layering, compartmentalizing, partitioning, etc.), and the like, where it is desirable to make such tablets, granules, layers, and/or sequestered components that possess enteric function and/or possess delayed release/admixing properties.

The delivery devices as described herein may be used to prepare liquids as free-flowing powders or compressed solids, to store a substance, to separate reactive substances, to reduce toxicity of a substance, to protect a substance against oxidation, to deliver a substance to a specified environment and/or to control the rate of release of a substance. In embodiments, the delivery devices as described herein may be used for delivering any of the loading substances described herein to a subject. Examples of subjects include mammals such as mice, rats, cows or cattle, horses, sheep, goats, and primates, including apes, chimpanzees, orangutans, fish, shellfish, crustaceans, birds (e.g., chickens, roosters, and the like), humans or domesticated animals (e.g., dogs and cats).

Also disclosed herein are foodstuffs comprising any of the delivery devices disclosed herein. By “foodstuff” is meant any article that may be consumed (e.g., eaten, drank, or ingested) by a subject. In embodiments, the delivery devices may be used as nutritional supplements to a foodstuff. For example, the delivery devices may be loaded with vitamins, omega-3 fatty acids, and other compounds that provide health benefits. In embodiments, the foodstuff is a baked good, a meat product, a frozen dairy product, a milk product, a cheese product, an egg product, a condiment, a snack food, a nut product, a plant protein product, a hard candy, a soft candy, a poultry product, a processed fruit juice, a granulated sugar (e.g., white or brown), a sauce, a gravy, a syrup, a nutritional bar, a beverage, a dry beverage powder, a jam or jelly, a fish product, or pet companion food.

In embodiments, provided are fills comprising any of the delivery devices described herein and a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as water, saline, and buffered solutions at physiological pH. Suitable carriers and their formulations are described in Remington, the Science and Practice of Pharmacy, 2005, 21^{st} edition, Lippincott Williams & Wilkins Company, Philadelphia, Pa., which is incorporated by reference herein for its teachings of carriers and pharmaceutical formulations. Typically, an appropriate amount of a pharmaceutically-acceptable salt is used in the formulation to render the formulation isotonic. Pharmaceutically-acceptable carriers are envisaged to have pH values from about 5 to about 8 and from about 7 to about 7.5. It will be apparent to those persons skilled in the art that certain carriers may be used depending upon, for instance, the route of administration and concentration of composition being administered. Other compounds may be administered according to standard procedures used by those skilled in the art.

Pharmaceutical formulations may include additional carriers, as well as thickeners, diluents, buffers, preservatives, surface active agents and the like in addition to the compounds disclosed herein. Pharmaceutical formulations may also include one or more additional active ingredients such as antimicrobial agents, anti-inflammatory agents, anesthetics, CNS drugs such as stimulants, depressants, antidepressants and the like.

The pharmaceutical formulation may be administered in a number of ways depending on whether local or systemic treatment is desired, and on the area to be treated. Administration may be vaginal, rectal, or oral.

Examples of non-aqueous solvents are propylene glycol, polyethylene glycol, fatty acids, fatty acid esters, mono-, di- or tri-glycerides of fatty acids, vegetable oils such as olive oil, fish oils, and injectable organic esters such as ethyl oleate. Aqueous carriers include water, alcoholic/aqueous solutions, emulsions or suspensions, including saline and buffered media.

Pharmaceutical formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Pharmaceutical formulations for oral administration include, but are not limited to, powders or granules, suspensions or solutions in water or non-aqueous media, capsules, sachets, or tablets. Thickeners, flavorings, diluents, emulsifiers, dispersing aids or binders may be desirable.

Some of the formulations may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, citric, tartaric, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The drug delivery devices as envisaged may be used to treat or prevent a variety of diseases depending upon the selection of the fill. For example, when the fill is an omega-3 fatty acid, the devices may be used to lower cholesterol levels, triglyceride levels, or a combination thereof in a subject.

The following examples are intended to illustrate but not limit the invention.

### EXAMPLES

### Example 1

### Gel Shell Matrix Elasticity Comparison

A. Gel Shell Matrix Elasticity without Reducing Sugar.

A 600 gram gel mass containing 28% (w/w) limed bone gelatin 150 bloom, 52% (w/w) water and 20% (w/w) glycerin was prepared by cooking gelatin in water and glycerin for 1.5 hours at 60° C. (Example 1A). The gel mass viscosity was measured using a Brookfield Viscometer after vacuum to eliminate air bubbles. The gel mass had a viscosity of 2000 centipoise (Cps) at 60° C.

The film was dried at room temperature and 1×2 inch dried rectangles were cut from the dried film to measure film elasticity (i.e., distance at break in millimeters) using a TA Texture Analyzer equipped with a double clamp set for tensile testing. Dried rectangular films expanded 133.27%.

B. Gel Matrices Tensile Strength with Reducing Sugar.

600 gram gel masses containing 28% (w/w) limed bone gelatin 150 bloom, 20% (w/w) glycerin, 3.75% (w/w) to 7.5% (w/w) fructose, and Q.S. to 100% with water were made by dissolving fructose in water, adding gelatin and glycerin, and cooking for 1.5 hr at 60° C. (Example 1B.1). Additional gel matrices were made containing 7.5% (w/w) (Example 1B.2) and 15% (w/w) fructose (Example 1B.3). Gel mass viscosities were measured as under Example 1A.

The resulting gel masses were cast into 0.04 inch films. The films were dried at room temperature and 1×2 inch dried rectangles were cut from the dried films to measure films' elasticity as described under Example 1A.

The results of gel mass viscosities and elasticity as defined by the % of film expansion before breaking are summarized in Table 1.

TABLE 1
Effect of fructose concentration on gel shell matrix viscosity and elasticity
of dried gelatin films.

| | Fructose | | | |
|---|---|---|---|---|
| | Concentration | Viscosity | Expansion | |
| Gel Matrix # | % W/W | (Cps) | (mm) | % Elasticity |
| 1A | 0.0 | 2,000 | 67.700 | 133.27 |
| 1B.1 | 3.75 | 2,040 | 82.686 | 162.77 |
| 1B.2 | 7.5 | 2,360 | 94.332 | 185.69 |
| 1B.3 | 15 | 3,680 | 95.561 | 188.11 |

As can be see in Table 1, soft gel matrices comprising fructose resulted in a gel films exhibiting greater elasticity over the same matrix in the absence of fructose. Given the negative affects expected in view of convention wisdom with respect to using non-reducing sugars in soft gel composition so as to avoid making the matrix mechanically weak (see, e.g., Gullapalli, J Pharmaceutical Sci (2010) 99(10):4107-4148 and Digenis et al., J Pharmaceutical Sci (1994) 83(7):915-921), the increased elasticity observed was, to say the least, counterintuitive if not unexpected.

C. Cortesi et al. Composition.

In support of the counterintuitive observation of increased tensile strength in the presence of a reducing sugar, a separate tensile strength assay was carried out with a sugar cross-linked gelatin composition as described by Cortesi et al., Biomaterials (1998) 19:1641-1649; 1642, “Preparation of gelatin disks”, col. 1, last paragraph bridging to col. 2, paragraph 1. Briefly, 15% (w/w) solution of 150 bloom bovine limed bone gelatin in water was mixed with 200 mg/ml fructose solution in a ratio of 3:1, respectively. The mixed gel was cast into 0.04 inch film and dried at room temperature for 72 hours followed by thermal treatment at 50° C. for 3 hours. The resultant films were exposed to the mechanical evaluation as in Example 1B, where the film composition of Cortesi et al. broke without any expansion. Further, a separate composition as described by Cortesi et al. using the treatment method as described (see, Cortesi et al. (1989), 1642, “2.5.1 Gelatin microspheres”, col. 1, paragraph 4), was cast and dried as described above. The resultant film had expanded by only 20% of the original size before breaking.

Cortesi et al. also described prolonged dissolution of gelatin microspheres made by a sequence of processes including thermal denaturing of a water-in-oil emulsion where the internal water phase contains gelatin and a native or oxidized sugar followed by dehydration using the organic solvent acetone. Such experimental approach seamed to prolong the dissolution time of gelatin microspheres and disks relative to those made by thermal treatment and dehydration in absence of sugars. However, this approach is not suitable to manufacture soft gelatin capsules as thermal treatment and dehydration destroy gelatin elasticity and cannot produce elastic films required to form soft gelatin capsules using a rotary die technology where their elasticity is maintained after drying. Films made according to the Cortesi et al. method and composition had zero elasticity. Even when plasticizers were added to the Cortesi et al. composition, dried gel films made with thermal treatment lost more than 80% of elasticity compared to the instant process. In fact, the thermal treatment at a minimum of 50° C., after drying, simply destroys capsules and can damage heat sensitive fill materials.

Based on these latter observations, the compositions of Cortesi et al. would support the expectation that the addition of reducing sugars makes “the matrix mechanically weak,” and further supports the notion that the addition of reducing sugars to gelatin-containing matrices to achieve increased elasticity/mechanical strength would not be expected.

### Example 2

### Gel Shell Matrix Comprising Insoluble Polymer, Elasticity Comparison

A. Gel Shell Matrix Containing Insoluble Polymer without Fructose.

A 600 gram gel mass containing 28% (w/w) limed bone gelatin 150 bloom, 39.6% (w/w) water, 8% (w/w) cellulose acetate phthalate (CAP), 3.2% (w/w) ammonia (10% w/v), 1.2% (w/w) triethyl-citrate and 20% (w/w) glycerin was made by first dissolving CAP in water/ammonia mixture, adding triethyl citrate followed by dissolving gelatin into the CAP solution and cooking for 1.5 hours at 60° C. (Example 2A). The gel mass viscosity was measure as above. Gel viscosity was 18,850 Cps.

The gel mass was cast to a 0.04 inch film and the elasticity was evaluated as described above. The dried rectangular films were able to expand by 89% before breaking.

B. Gel Shell Matrices Containing Insoluble Polymer and Fructose.

A 600 gram gel mass containing 28% (w/w) limed bone gelatin 150 bloom, 3.75% (w/w) fructose, 40.25% (w/w) water, 8% (w/w) CAP, 1.2% (w/w) triethyl citrate, 3.2% (w/v) of diluted ammonia (10% w/v), and 20% (w/w) glycerin was made by first dissolving CAP in water/ammonia mixture. Dissolving fructose and gelatin into the CAP solution and cooking for one hour at 60° C. before adding glycerin and continuing cooking for an additional 30 minutes at 60° C. (Example 2B.1). Additional matrices were made using 5.5% (w/w) (Example 2B.2) and 7.5% (w/w) (Example 2B.3) fructose. The viscosity values of the gel masses were measured as under Example 1A.

The gel masses were cast into 0.04 inch films and the elasticity values of the dried films were measured as described under Example 1A. The results of gel mass viscosity values and % elasticity of the dried films are provided in tabular form below (Table 2).

TABLE 2
Effect of fructose concentration on gel mass viscosity and elasticity of
dried gel films containing gelatin and acid insoluble polymer.

| | Fructose | | | |
|---|---|---|---|---|
| | Concentration | Viscosity | Expansion | |
| Gel matrix # | % W/W | (Cps) | (mm) | % elasticity |
| 2A | 0.0 | 18,850 | 45.252 | 89.08 |
| 2B.1 | 3.75 | 23,000 | 50.165 | 98.75 |
| 2B.2 | 5.5 | 33,260 | 61.240 | 120.55 |
| 2B.3 | 7.5 | over 40,000 | 58.266 | 114.70 |

As can be seen in Table 2, similar to the results using soft gel matrix without the insoluble polymer, the soft gel matrices comprising fructose resulted in gel films which exhibit greater elasticity over the same matrix in the absence of fructose.

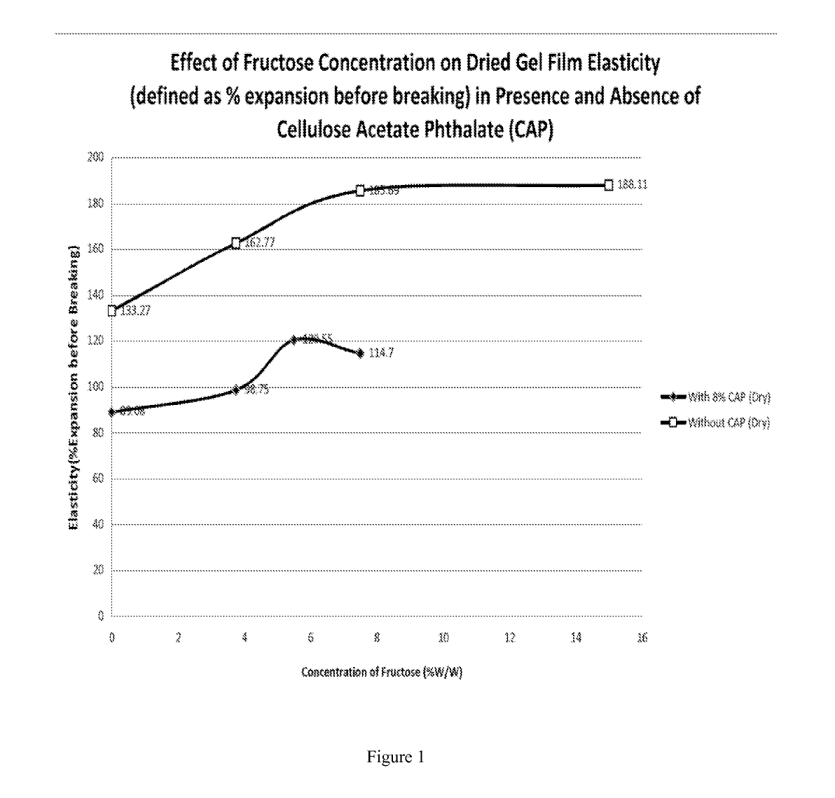

FIG. 1 summarizes the findings in Examples 1 and 2, showing in graphic form the improvement of gelatin film elasticity when fructose is added at different concentrations to the gel shell matrix.

### Example 3

### Dissolution of Fructose Containing and Non-Fructose Containing Gel Matrices in 0.1N HCl

1×2 inch rectangles of dried gel films of gel matrices 1A (no fructose, no CAP), 1B.1 (with 3.75% w/w fructose, no CAP), 2A (no fructose, 8% w/w CAP) and 2B.1 (3.75% fructose, 8% w/w CAP) were tested for dissolution in 0.1N HCl at 37° C. using USP apparatus II at 50 rpm. Films 1A and 1B.1 distorted and melted down into a ball in three minutes. Film 2A resisted acid for 30 minutes and ruptured and eroded after one hour. Film 2B.1 remained intact and flat after 2 hours.

100 kg of the gel mass as described in Example 2(B) was manufactured and used in making soft gelatin capsules, each containing 1000 mg of fish oil, 15 mg of peppermint oil, 2.5 mg of fennel oil and 2.5 mg of ginger oil. The dried capsules had burst strength of more than 55 kg as tested by TA Texture Analyzer equipped with a flat probe. The dried capsules resisted the mechanical and acid stress of 0.1N HCl at 37° C. using standard USP disintegration apparatus for one hour.

In addition, the Cortesi et al. composition was also tested for acid resistance as described above. It was observed that the Cortesi et al. composition deformed and completely dissolved in 0.1N HCl in five minutes.

### Example 4

### Disintegration Test

A 100 kg gel mass containing 28% (w/w) limed bone gelatin 150 bloom and 5.5% (w/w) fructose, 38.5% (w/w) water, 8% (w/w) CAP, 1.2% (w/w) tri-ethyl citrate, 3.2% (w/w) diluted ammonia (10% w/v), and 20% (w/w) glycerin was made as a capsule comprising the various oils as described above. The gel mass viscosity was measured as described in Example 1. The dried capsules resisted the mechanical and acid stress of 0.1N HCl at 37° C. using standard USP disintegration apparatus for 1.3 hours yet dissolved in pH 6.8 buffer preheated to 37° C. within 10 minutes. In addition, the capsules were tested according to USP disintegration test procedure using USP simulated gastric fluid containing pepsin. Capsules were intact after one hour in acidic environment and disintegrated in pH 6.8 buffer within 5 minutes.

Based on these observations, it has been discovered that, despite well know assertions relating the disadvantages of using fructose and glucose, including their incompatibility with gelatin in soft gel capsules, these reducing sugars provide unexpected advantages to gelatin films both in terms of elasticity and mechanical strength.

While not being bound by theory, the presence of reducing sugars appear to have exceptional plasticizing effect on the gelatin-acid insoluble polymer mixture, but did not render the gel mass un-sealable or rubbery as expected from normal cross-linking reactions associated with known crosslinking agents such as formaldehyde.

Although the invention has been described with reference to the above examples, it will be understood that modifications and variations are encompassed within the spirit and scope of the invention. Accordingly, the invention is limited only by the following claims. All articles and patent publications recited are herein incorporated by reference in entireties.

We claim herein:

1. A soft gel shell matrix comprising:
a) a water soluble film former;
b) an acid insoluble polymer;
c) a reducing sugar; and optionally
d) a plasticizer.

2. The soft gel shell matrix of claim 1, wherein the water soluble film former is selected from the group consisting of gelatin, cellulose derivatives, modified starches, natural or synthetic polymers, and combinations thereof.

3. The soft gel shell matrix of claim 1, wherein the water soluble film former is gelatin.

4. The soft gel matrix of claim 3, comprising about 15 to 55% (w/w), about 18 to 45% (w/w), or about 20 to 40% (w/w) gelatin.

5. The soft gel shell matrix of claim 4, comprising about 25% (w/w) gelatin.

6. The soft gel shell matrix of claim 1, wherein the reducing sugar is glucose, fructose or a combination thereof.

7. The soft gel shell matrix of claim 6, comprising a reducing sugar at about 0.5 to 20% (w/w), at about 2 to 15% (w/w), or at about 3 to 10% (w/w).

8. The soft gel shell matrix of claim 6, wherein the reducing sugar is fructose.

9. The soft gel shell matrix of claim 8, comprising about 3.75% (w/w) fructose.

10. The soft gel shell matrix of claim 1, wherein the acid insoluble polymer is selected from the group consisting of cellulose acetate phthalate (CAP), cellulose acetate trimellitate (CAT), cellulose acetate succinate (CAS), hydroxypropyl methyl cellulose phthalate (HPMCP), hydroxypropyl methyl cellulose acetate succinate (HPMCAS), carboxy methyl ethyl cellulose (CMEC); pectin, alginate; copolymers of methacrylic acid and methyl methacrylate, ethyl acrylate; terpolymers of methacrylic acid, methacrylate, ethyl acrylate, and polyvinyl acetate phthalate (PVAP).

11. The soft gel shell matrix of claim 10, wherein the plasticizer is selected from the group consisting of sorbitol, glycerol, polyethlene glycol, poly-alcohols with 3 to 6 carbon atoms, citric acid, citric acid esters, triethyl citrate, and combinations thereof.

12. The soft gel shell matrix of claim 1, wherein the matrix comprises a water soluble film former at about 15 to 55% (w/w), an acid soluble polymer at about 0.5 to 30% (w/w), a reducing sugar at about 0.5 to 20% (w/w), a plasticizer at about 5 to 30% (w/w), and about 10 to 40% (w/w) water.

13. The soft gel shell matrix of claim 12, further comprising additives selected from the group consisting of flavors, sweeteners, colorants, preservatives, fill, and combinations thereof, wherein said matrix possesses elastic properties such that the matrix forms a soft gel capsule via rotary die technology.

14. An acid resistant soft gel capsule comprising a water soluble film former; an acid insoluble polymer; a reducing sugar; a plasticizer; water and a fill, wherein the fill comprises a pharmaceutical composition, food supplement, or a nutraceutical composition.

15. The capsule of claim 14, wherein the water soluble film former is gelatin.

16. The capsule of claim 15, wherein the gelatin has between about 50 to 275 blooms.

17. The capsule of claim 14, wherein the reducing sugar is fructose or glucose.

18. The capsule of claim 17, comprising fructose at between about 0.5 to 20% (w/w).

19. The capsule of claim 18, comprising about 3.75% (w/w) to about 5% (w/w) fructose.

20. The capsule of claim 17, wherein the pharmaceutical composition is selected from the group consisting of diclofenac sodium, hydroxyethyl pyrrolidine diclofenac, diethylammine diclofenac, ibuprofen, flurbiprofen, ketoprofen, idomethacin, mefenamic acid, naproxene, nimesulide, piroxicam, amiodarone, diisopyramide, verapamil, propranolol, amoxicillin, flucloxacillin, gentamicin, rifampicin, erythromicine, cephalosporin, amfotericin, buconazol nitrate, ketoconazol, econazol, fluconazole, flucitosina, griseofulvine. itraconazol, miconazol, rystatin, sulconazol, tioconazol, acyclovir, gancyclovir, AZT, protease inhibitor, amlodipine, clonidine, diltiazem, felodipine, guanabenz acetate, isradipine, minoxidil, chloride nicardipine, nifedipine, chloride prazosin, papaverine, carbamazepine, difenidramine, chlorfeniramine, chlorciclizine, prometazin, acrivastine, loratadine, terfenadine, cyclosporine, decarbazine, etoposide, lomustine, melphalan, mitomicin, mitoanthrone, procarbazine, taxol and derivatives thereof, alprazolam, bromazepam, diazepam, lorazepam, oxazepam, temazepam, sulpiride, triazolam, alprenolol, atenolol, oxprenolol, pindolol, propranolol, salbutamol, salmeterol, aminone, digitoxinn, digoxin, lanatoside C, medigoxine, ubidecarenone, bechlomethasone, betamethasone, budesonide, cortisone acetate, desossimetasone, dexamethasone, fludrocortidone acetate, flunisolide, hydrocortisone, methylprednisolone, prednisone, triamicinolone, cimetidine, cisapride, domperidone, famotidine, loperamide, mesalazine, omeprazol, ondansetron, ranitidine chloride, bezafibrate, chlorofibrate, gemfibrozil, probucol, amil nitrate, gliceryltrinitrate, isosorbide dinitrate and mononitrate, pentaeritritol tetranitrate, Nicotina, codeine, destropropoxifene, dihydrocodeine, morphine, pentazocine, methadone, danazol, ethynilestradiol, medroxyiprogesterone acetato, methyltestosterone, testosterone, noretistrone, norgestrel, estradiolo, estriolo, progesterone, stilbestrolo, diethylstilbestrol, peptides, peptidic molecules, and combinations thereof.

21. The capsule of claim 17, wherein the nutraceutical composition is selected from the group consisting of artichoke, bilberry, bioflavonoid, boswella, bupleurium, chamomile, chlorophyll, cranberry, damiana, echinacea, essiac, garcinia cambogia, garlic, germanium, ginger, gingko, ginseng, goldenseal, grape seed, green tea, hawthorne berry, hesperidin, hops, horse chestnut hydrangea, hypericum, indole-3-carbinol, licorice, lycopene, nettle root, peppermint, periwinkle, policosanol, psyllium, pygeum, quercetin, raspberry, resveratol, rutin, sassafras, saw palmetto, silymarin, tribulus terestris, turmeric, valerian, wild yarn, acetyl-1-carnosine, alpha lipoic acid, amylase, androstendiol, androstendione, arginine, ascorbic acid, B vitamin, β-carotene, biotin, bromelain, calcium, chicken collagen, chitosan, choline, chondroitin, coenzyme Q10, creatine, dehydroepiandrosterone, diethylmethylaminoethanol, dihydroepiandsterone, dimethylglycine, DMSO, gammahydroxybutric acid (GABA), glucosamine, glutamine, glutathione, hyaluronic acid, hydroxytryptophan, indium, isoleucine, 1-carnitine, lactoferrin, lecithin, leucine, lipase, lumbrokinase, lutein, magnesium, melatonin, Methylcobalamin, methylsulfonylmethane, MGN 3, ornithine, pancreatin, panthethoic acid, papain, para-amino benzoic acid (PABA), phenylalanine, phosphatidylcholine, phosphatidylserine, potassium, pregnenalone, protease, retinoic acid, retinol, s-adenosyl-methionine, selenium, taurine, theanine, thymase, tocopherol, trimethylglycine, tryptophan, tyrosine, valine, vinpocetine, vitamin D, vitamin A, zeathanthine, zinc and combinations thereof.

22. A process of manufacturing a shell composition into soft capsules comprising:
a) preparing a solution comprising a water soluble film former, an acid-insoluble polymer, a reducing sugar and mixing with one or more plasticizers to form a gel mass;
b) casting the gel mass into films or ribbons using heat-controlled drums or surfaces; and
c) forming a soft capsule using a rotary die device,
wherein the manufactured soft capsule has a burst strength of greater than about 7 kg and resists acid stress at about 0.1N HCl for at least about 30 minutes or at least about one hour.

23. The process of claim 22, wherein the thickness of the films or ribbons is from about 0.015 inches to about 0.050 inches.

24. The process of claim 21, wherein the thickness of the films or ribbons is about 0.030 inches.

25. The process of claim 22, wherein the manufactured soft capsule disintegrates at pH 6.8 in less than about 20 minutes or less than about an hour.

26. An enteric or delayed release delivery device for administration of a nutraceutical composition, a pharmaceutical composition, food, or food supplement to a subject in need thereof produced by the process of claim 22.

27. The delivery device of claim 24, wherein the reducing sugar is fructose or glucose.

28. A coating comprising:
a) a water soluble film former;
b) an acid insoluble polymer;
c) a reducing sugar; and optionally
d) a plasticizer;
wherein said reducing sugar does not render the resulting film un-sealable.

29. The coating of claim 28, wherein said coating covers a tablet, powder, solid or semi-solid comprising a material that is sensitive to acidic pH, and wherein said material is to be released into the intestine.

30. The coating of claim 29, wherein the reducing sugar is fructose.
